(19) United States
(12) Patent Application Publication
(10) Pub. No.: US 2011/0230352 A1
(43) Pub. Date: Sep. 22, 2011

(54) TREATMENT PROCESS FOR A SUPERIOR PLANT IN ORDER TO CONTROL ITS GROWTH AND ARCHITECTURE

(75) Inventors: Catherine Rameau, Versailles (FR); Jean-Paul Pillot, Versailles (FR); Guillaume Becard, Odars (FR); Victoria Gomez-Roldan, Toulouse (FR); Virginie Puech-Pages, Toulouse (FR); Francoise Rochange, Ramonville St Agne (FR); Christine Beveridge, Brisbane (AU); Elizabeth Dun, Brisban (AU); Phil Brewer, Taringa (AU)

(73) Assignees: INSTITUT NATIONAL DE LA RECHERCHE AGRONOMIQUE, Paris Cedex 07 (FR); UNIVERSITE PAUL SABATIER, Toulouse Cedex (FR); UNIVERSITY OF QUEENSLAND, St Lucia (AU)

(21) Appl. No.: 12/989,215

(22) PCT Filed: Apr. 21, 2009
(86) PCT No.: PCT/FR2009/050738

§ 371 Date: Jun. 1, 2011

(30) Foreign Application Priority Data
Apr. 23, 2008 (FR) 08 52737

(51) Int. Cl. A01N 43/08; C07D 307/93; A01P 21/00

(52) U.S. Cl. 504/297; 549/299

(57) ABSTRACT

A treatment process for a superior plant in order to control the growth of the plant, is characterized in that an adapted quantity of strigolactones is brought in contact with the plant so as to inhibit the formation of at least one ramification. A method of using strigolactones to identify genes and/or molecules involved in the growth of buds and/or ramifications in superior plants is also disclosed.

### TECHNICAL FIELD

The invention relates to a treatment process in order to control the growth and architecture of superior plants. More precisely, the invention relates to the use of strigolactones for selectively or totally inhibiting bud growth of a plant of interest, and thus the number of ramifications. The inhibition can be temporary so as to control the growing period of theses buds, or permanent in order to facilitate the growth of other ramifications to the detriment of the inhibited one(s). The invention also relates to the use of strigolactones for identifying genes and/or molecules intervening in the control process for the growth and sprouting of buds and/or ramifications of superior plants.

The invention finds applications in the agricultural field, for cultivating food plants, leguminous plants, forest plants, ornamental plants etc, for which the control of the ramification number and/or the ramification period can improve the yield and/or the production quality (fruit size, quality of wood etc.). The expression “Superior plants” means pluricellular vascular plants with roots and an aerial part. The term used “Cultivation” means field cultivation as well as forest cultivation and in vitro cultivation, soilless cultivation or other.

### STATE OF THE ART

Plants that are cultivated for their flowers, their fruits, seeds or vegetative parts, are submitted to many control and treatment processes, so as to get the best yield and the best quality.

Thus, for example, it is attempted to control the flowering periods so as to prevent the floral buds from appearing during periods having a high risk of frost. In the same manner, when it is desired to obtain large-grade fruits, or more generally more robust plants, the plant is pruned so as to limit the number of ramifications and thus the number of “well-like” organs represented by fruits in the growth period or seeds in the filling period. The use of fertilizers also enables a yield optimization.

In such control and treatment processes, it is necessary to know, besides the plant itself, the conditions in which it is cultivated: soil nature, climate etc, notably to know when and how the plants must be pruned. Furthermore, pruning is a manual process which is fastidious and expensive and needs the intervention of skilled persons.

### PRESENTATION OF THE INVENTION

The objective of the invention is to provide a new treatment process for plants which enables to control their growth by totally or partially inhibiting in a definitive or temporary manner the growth of ramifications, notably so as to optimize the yield of theses plants.

Therefore, in the invention, it is proposed to bring the plants to be treated into contact with strigolactones so as to inhibit or limit the sprouting of all or part of the ramifications.

Strigolactones are molecules composed of a tricyclic lactone connected to a butyrolactone ring by an enol ether bridge.

Many natural or synthetic strigolactones are currently known.

Notably, in the document FR2865897, several strigolactones are used to amplify the development and/or growth of arbuscular micorhizal fungi so as to increase the symbiotic interaction between these microorganisms and host plants.

Strigolactones are also known as germination inductors for seeds of parasitic plants such as Orobanches. In order to eliminate such plants from agricultural soils, said soils are treated with strigolactones so as to induce the germination of parasitic plants in the absence of host plants, which leads to their death through shortage of nutrition.

The inventors have surprisingly discovered that strigolactones also intervene in the growth of superior plants by controlling the start of ramifications and would correspond to the ramification repressor signal SMS (Shoot Multiplication Signal) identified in several species dicotyledon and monocotyledon through the characterization of hyper-ramified mutants, notably the pea mutants rms1 to rms5 (Beveridge 2006).

The object of the invention is thus a treatment process for a superior plant in order to control the growth and architecture of the plant, characterized in that an adapted amount of strigolactones are brought into contact with the plant so as to inhibit the formation of at least one ramification.

The strigolactones used are natural strigolactones such as

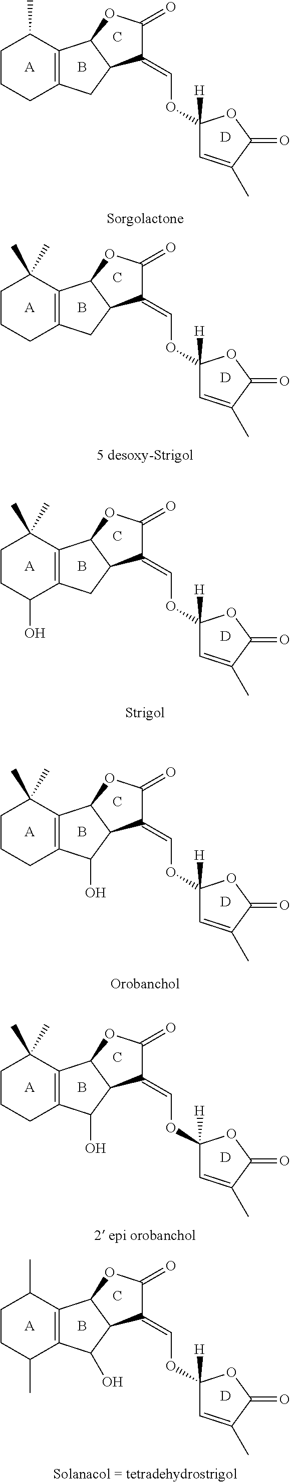

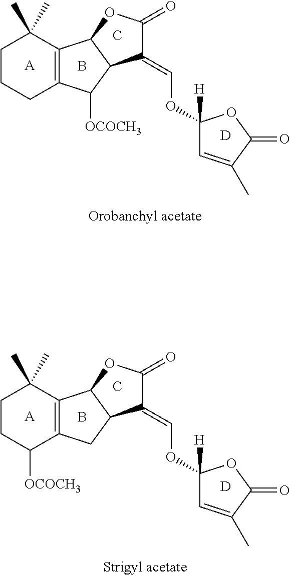

as well as synthetic strigolactones, such as GR24, or the molecule ABC, comprising only some rings (A, B and C) of stringolactones:

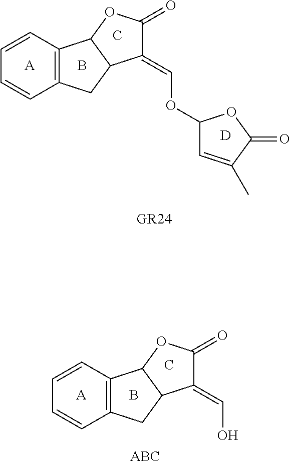

The term used “to inhibit” means to repress the growth of a bud in a definitive or temporarily manner. Thus, according to the invention, a ramification can be suppressed by definitely inhibiting the growth of the corresponding bud or bringing said bud into a dormancy state so as to delay its growth.

The term used “ramification” means the development from the axillary bud under the leaf axil, whether it is a branch, a flower or an inflorescence.

The inhibition can be total, i.e. it concerns all the axillary buds when treating the plant, or targeted, i.e. it concerns only the buds that must be specifically treated.

The treated plants can be cultivated under glass as well as with soil, in vitro or even soilless.

The expression “an adapted amount” means an amount which is at least sufficient for having an action on the growth and architecture of the plant to be treated.

According to the invention, a solution containing strigolactones can be applied onto an at least partial portion of the aerial part of the plant. For example, the composition can be sprayed or deposited onto the buds the repression of which is desired, or onto the part of the plant the growth of which is desired to be controlled. It is otherwise possible to inject the composition at the buds themselves, or at the stems carrying the buds to be repressed.

In another example of the process according to the invention, it is possible to enrich the soil with strigolactones, so as to reduce the number of stems in a non-selective manner or to slow down their growth. The inventors has indeed observed that the repression signal SMS for the growth of ramifications migrates in the direction root-stem, so that it is conceivable that this signal is carried by the crude sap in the xylem (Food et al. 2001 Pl Physiol. 126:203-209).

Advantageously, the strigolactone concentration in the composition is at least of 1 nM and will vary according as it is desired to inhibit the bud growth in a definitive or temporary manner, the concentration further depending on the nature of the plant to be treated. Generally, the strigolactone concentration to be used will vary between 1 nM and 100 μM, and preferably between 100 nM and 1000 nM.

The number of treatment days can also vary according to the plant, its age at the time of the treatment, the final or non-desired effect etc.

An object of the invention is also to use strigolactones for identifying genes and/or molecules intervening in the control of the growth of buds and/or ramifications in superior plants.

Thus, strigolactones can be used for identifying strigolactone receptors in plants. The gene RMS4, which is supposed to be involved in the response to the signal SMS, encodes an F-box protein. But there are several examples of vegetal hormone receptors which are F-box proteins: the auxine receptor TIR1 (Dharmasiri et al. 2005 Nature 435:441-445); the jasmonic acid receptor COI1 (Xie et al. 1998 Science 280:1091-1094).

Strigolactones can also be used for identifying components in the signaling pathway by screening mutants resisting to strigolactones. Natural or synthetic strigolactones, such as GR24, can be used for screening mutants that resist to strigolactones and/or do not respond to the application of strigolactones. The genes corresponding to mutants are then cloned so as to identify new proteins in the signaling pathway (Leyser et al. 1993 Nature 364: 161-164; Guzman and Ecker 1990 Plant Cell. 6:513-523).

It is also possible to use strigolactones for identifying components in the signaling pathway by identifying genes the expression of which is modified, i.e. repressed or induced, par applying strigolactones (Ulmasov et al. 1997 Sciences 276:1865-1868; Thines et al. 2007 Nature 448:661-665).

It is also possible to identify more stable chemical analogs having the same biological activity as the natural strigolactone molecules, for example with a lower fabrication cost. Insofar as strigolactones have an action on several processes (mycorhyzation, germination of parasitic seeds, ramification), it is possible to identify and fabricate molecules with activities specific to the various processes by identifying patterns which are essential to each biological activity, in a manner similar to the identification of synthetic analogs realized for the main phytohormones such as NAA, IBA or 2,4-D (synthetic auxines), kinetine (synthetic cytokinine).

It is otherwise possible to use strigolactones for identifying all or part of its agonists or antagonists, i.e. molecules able to positively or negatively modulate the response to strigolactones as that has been described for the identification of agonists and antagonists of auxine (Hayashi et al. 2008 PNAS 105:5632-5637).

### BRIEF DESCRIPTION OF THE FIGURES

FIGS. 1A and 1B show the results of the qualitative and quantitative analysis of the majority strigolactone in the root exudates in wild pea and in the mutants rms1 and rms4;

FIG. 2 represents a bar chart illustrating the effect of synthetic strigolactone GR24 applied onto pea mutants;

FIG. 3 represents a bar chart illustrating the effect of various synthetic and natural strigolactones on pea mutants;

FIG. 4 represents a bar chart illustrating the effect of synthetic strigolactone GR24 applied onto a wild pea;

FIGS. 5A and 5B represent charts illustrating the effect of synthetic strigolactone GR24 according to the development stage (size) of the buds onto which it has been applied;

FIG. 6 represents a bar charts illustrating the effect of the synthetic strigolactone GR24, injected into pea mutants at increasing concentrations, on the start of the bud situated at a distance above the injection zone;

FIGS. 7A, 7B and 7C represent bar charts illustrating the decapitation effect for the plant on an axillary bud which has been first inhibited by strigolactone (FIGS. 7A and 7B) and of strigolactone on axillary buds of a decapitated plant (FIG. 7C);

FIG. 8 represents a bar chart showing the absence of effect of strigolactone on the apical bud in wild pea;

FIG. 9 represents a bar chart illustrating the effect of synthetic strigolactone GR24 on wild and mutant plants Arabidopis thaliana;

FIG. 10 represents a bar chart illustrating the effect of strigolactones, applied through the roots, on the internodal length in wild pea (line WT Térèse) and in mutants (line M3T-988 ccd8/rms1 from WT Térèse);

FIG. 11 represents a bar chart illustrating the effect of strigolactones, applied through the roots, on the ramification length in wild pea (WT Térèse) and in mutant (line M3T-988 ccd8/rms1 from WT Térèse).

### DETAILED DESCRIPTION OF THE INVENTION

The objective is to test the effect of synthetic strigolactones GR24 and of natural strigolactones on the hyper-ramified mutants ramosus (rms) in pea (Beveridge 2000 Plant Growth Regulation 32:193-203). Mutants rms are known as having a number of ramifications which is much higher than the number of ramifications in wild pea and notably at all the plant nodes.

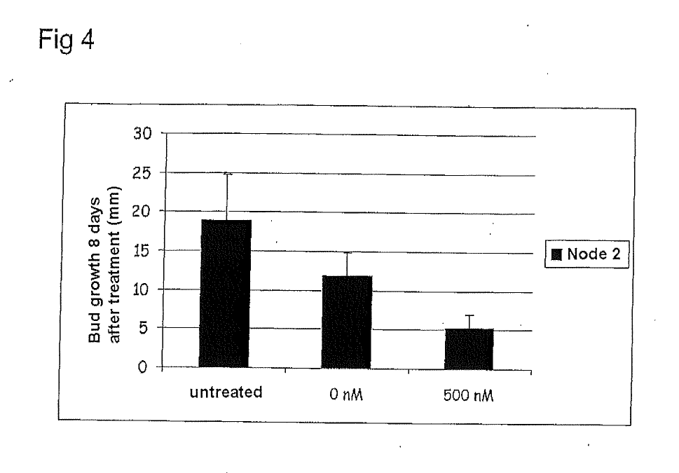

These mutants rms have been obtained from different wild genetic funds (WT) which have axillary buds which are generally dormant. These peas WT can however ramify at first two plant nodes according to environmental conditions and different experiments have been carried out on wild peas (WT Térèse—FIG. 4).

Generally, in peas, the first two scales are considered as the first two nodes, the cotyledonary node being the node 0.

The detailed characterization of hyper-ramified pea mutants rms allowed to reveal the existence of a new signal called SMS (Beveridge 2006) repressing the plant ramification:
the mutants rms1 and rms5 are biosynthetic mutants of the signal SMS. The ramification of these mutants is repressed when the mutant stem is grafted onto a wild stock (Morris et al. Pl Physiol. 126:1205-1213). The genes RMS1 and RMS5 both encode for Carotenoid Cleavage Dioxygenase (Sorefan en al. 2003 Genes Dev 17:1469-1474; Johnson et al. 2006 Plant Physiol 142:1014-1026), which suggests that the signal SMS is a derivate of cartenoids such as strigolactones (Matusova et al. 2005 Plant Physiol 139:920-934). These genes are kept in plants and homologues have been identified in rice, petunia or poplar. Thus, the pea gene RMS5 corresponds to the Arabidopsis gene MAX3 and to the rice gene HTD1 (Johnson et al. 2006 Plant Physiol 142:1014-1026). It is thus supposed that the signal SMS is kept in plants.
the mutant rms4 is involved in the reception or the signaling pathway of the ramification repressing signal: the ramification of this mutant is not repressed when the mutant stem is grafted on a wild stock (Beveridge et al. 1996 Plant Physiol 110:859-865).

In the plant Arabidopsis thaliana, another gene of the biosynthesis pathway for the signal SMS has been identified: i.e. the gene MAX1. The corresponding enzyme MAX1 (a cytochrome PA450) seems to intervene before both Carotenoid Cleavage Dioxygenases RMS1/CCD8 and RMS5/CCD7.

The inventors have showed that an already known molecule family, the strigolactone family, could be used for repressing the growth of axillary buds of a plant. These results suggest that the signal SMS identified with the help of hyper-ramified pea mutants rms would belong to the strigolactone family.

In order to check this hypothesis, the inventors have searched and quantified the strigolactones produced by the wild pea or the mutants.

Firstly, the authors have searched strigolactones in root exudates of the wild pea WT Térèse.

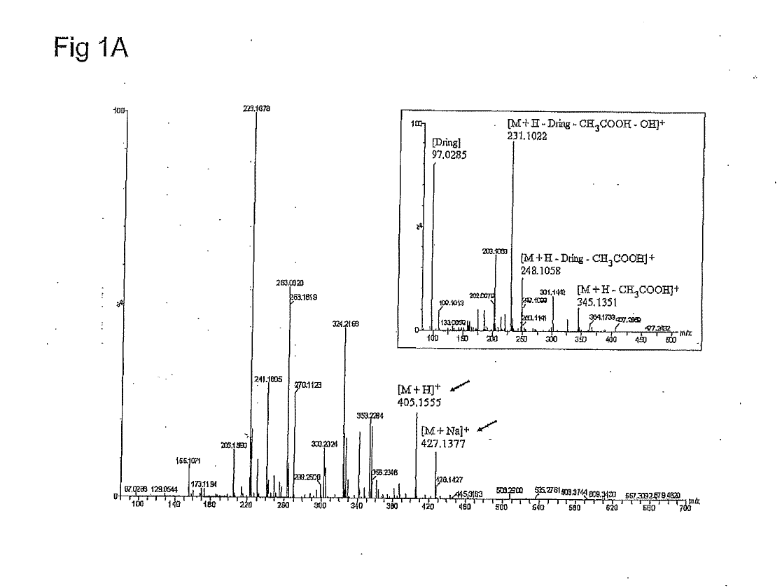

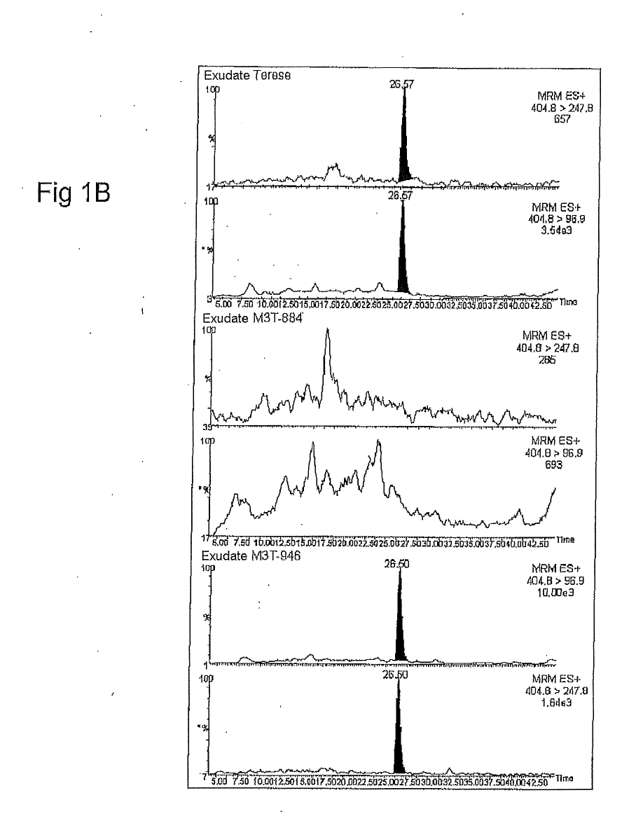

To this end, they have analyzed the exudate extract ethyl acetate by using a high resolution mass spectrometry on HPLC/QTOFMS (Ultra-Performance Liquid Chromatography coupled to a Quadrupole Time-Of-Flight). When searching parent ions able to generate a produced ion at m/z:97.0285, corresponding to the cycle D, common to all the characterized strigolactones, they observed a majority peak on the chromatogram. The spectrum obtained for this component presents the ions m/z 405.1555 and m/z 427.1377 (FIG. 1A), respectively corresponding to the theoretical mass of a molecule having the general formula C₂₁H₂₅O₈[M+H]⁺ and C₂₁H₂₄O₈Na[M+Na]⁺. The general formula C₂₁H₂₄O₈ could correspond either to a strigyl acetate or to a orobanchyl acetate carrying the supplemental group hydroxyl or epoxy. This identity is confirmed by all the produced ions observed by MS.MS: the ions m/z 345.1351 [M+H—CH₃COOH]⁺, 248.1058 [M+H—cycle D—CH₃COOH]⁺ and 97.0285 [cycle D]⁻ (FIG. 1A).

This analysis enables to confirm the presence of a new strigolactone in pea exudates (Térèse), the exact structure of which has not been determined yet.

Subsequently, the inventors have quantified the quantity of this strigolactone in the exudates of wild pea from Térèse, mutants rms1 line M3T-884 from Térèse and rms4 line M3T-946 from Térèse.

The spectra in FIG. 1B correspond to fragmentations of the majority strigolactone with loss of cycle D+acetate (spectrum 404.8>247.8) and with loss of cycle's ABC (spectrum 404.8>96.9). it is observed that this strigolactone, present in the root exudates of wild pea (Térèse), is present in the mutant rms4 (line M3T-946), but is not detectable in the mutant rms1 (line M3T-884).

### Example 1

### Test of Hyper-Ramification Pea Mutants and of Wild Peas with Local Application of Strigolactones for Demonstrating the Effect by Direct Application of Strigolactones

A] Experiment No 1

A first experiment is carried out simultaneously on mutants rms1 (line M3T-884 from wild pea WT Térèse) and rms4 (line M3T-946 from wild pea WT Térèse).

9 seeds are used for each treatment; they are sowed in pots (3 plants per pot) in soil mixed to clay balls. The seedling is carried out under glass with a light period of 16 h light/8 h night.

The treatment is carried out 10 days after the seedling (Stage with 4 leaves).

A solution containing synthetic strigolactone GR24 dissolved in acetone at 0 nM and 100 nM (4% PEG 1450, 25% ethanol, 5⁰/₀₀ acetone) is applied with the help of a micropipette onto the buds at the node 4 (N4), at the rate of 10 μl per bud.

The buds and/or ramifications at the first two plant nodes N1 and N2 are cut for favoring the start of the buds at upper nodes.

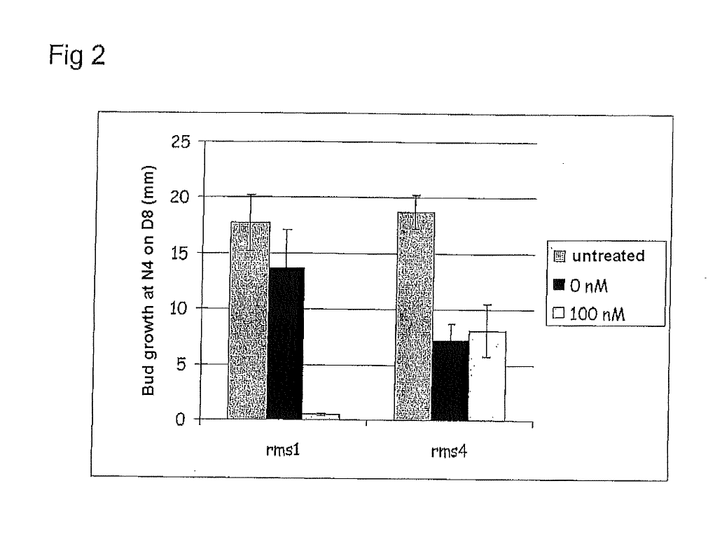

The chart in FIG. 2 shows the results of the bud growth at N4 (the bud size on the treatment day—the size bud on the 8^{th} day) obtained eight days after the treatment.

The untreated plants correspond to the plants the buds and/or ramifications of which at nodes 1 and 2 have been cut but which have not received any treatment. The control “0 nM” corresponds to the plants treated with the same solution as for the treatment “500 nM” but without any strigolactones.

It is noted that the bud growth at N4 in the mutant ms1 is strongly repressed by the treatment at 100 nM, whereas the buds of the mutant rms4 are not repressed in a significant manner, which is in accordance with the results expected with the signal SMS. The vegetal hormone repressing the ramification in superior plants is probably a molecule of the strigolactone family.

The application of synthetic strigolactone GR24 directly onto axillary buds enable to inhibit the growth of said treated buds in the mutant rms1.

B] Experiment No 2

A second experiment is carried out on mutants rms1 (line M3T-884 from wild pea WT Térèse) in order to compare on these mutants the effect of synthetic strigolactone GR24, of a synthetic molecule ABC from GR24, without any cycle D characteristic of strigolactone, and Sorgolactone, a natural strigolactone.

The plants used are obtained in the same manner as for the plants used in the first experiment.

The solution containing synthetic strigolactone GR24 at 0 nM, 100 nM and 500 nM (4% PEG 1450, 10% ethanol) is applied with the help of a micropipette onto the buds at the node 4 (N4), at the rate of 10 μl per bud.

The buds and/or ramifications at the first two plant nodes N1 and N2 are cut at the time of the treatment.

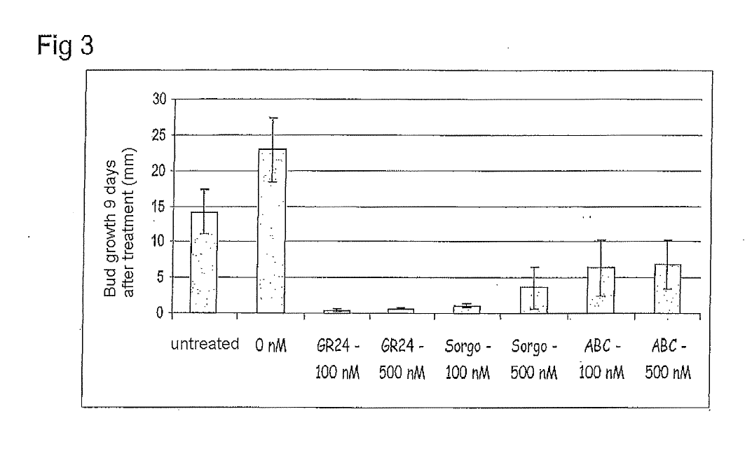

The size of the buds at the upper nodes (node N4) is measured nine days after the treatment. The results of the bud growth are illustrated in the chart in FIG. 3.

It is noted that GR24 and sorgolactone have comparable effects, the difference observed in the chart at 500 nM being due to a statistic effect because of the small number of tested plants (8 or 9 plants). All the strigolactones enable to inhibit in a significant manner the growth of treated buds right from 100 nM. The molecule ABC seems to be less efficient than GR24 and sorgolactone.

C] Experiment No 3

We tried to prove the effect of strigolactones on wild peas.

To this end, a third experiment is carried out on wild peas WT Térèse. The plants used are obtained in a identical manner as for the plants used in the first experiment.

The treatment is carried out 10 days after the seedling (stage with 4 to 5 leaves).

A solution containing synthetic strigolactone GR24 at 0 nM and 500 nM (4% PEG, 10% ethanol) is applied with the help of a micropipette onto the buds at the node 2 (N2), at the rate of 10 μl per bud.

The buds and/or ramifications at the first plant node N1 are cut at the time of the treatment.

The bud size at the node N2 is measured eight days after the treatment, the results being plotted in the chart in FIG. 4.

It is noted that synthetic strigolactone GR24 has also an action on the growth of the buds treated by local application in wild pea.

### Example 2

### Test of Hyper-Ramification Pea Mutants with Local Application of Strigolactones at Different Stages of the Bud Growth

The objective is to study the effect of strigolactones on the start of axillary buds according to the size and/or the development stage of the bud at the time of the treatment.

A] Experiment No 1

A first experiment is carried out on mutants rms1 (line WL5237 from wild pea WT Parvus) in order to compare the effect of synthetic strigolactones GR24 on the size of the treated buds at the time of the treatment.

In this experiment, 20 seeds are used for each treatment, which are sowed in pots (2 plants per pot with a diameter of 15 cm) in a soil mixed to clay balls. The seedling is carried out under glass with a natural light and with an extension of the light period of 18 h light/6 h night by means of incandescent bulbs (60 W).

On the first day of treatment, a solution containing synthetic strigolactone GR24 at 0 nM and 1000 nM (4% PEG 1450, 10% ethanol) is applied with the help of a micropipette onto the buds at the node 3 (N3), at the rate of 10 μl per bud.

On the second day of treatment, a solution containing synthetic strigolactone GR24 at 0 nM and 1000 nM (4% PEG 1450, 10% ethanol) is applied with the help of a micropipette onto the same buds, at the rate of 10 μl per bud.

The buds and/or ramifications at the first two plant nodes N1 and N2 are cut at the time of the treatment.

The first treatment is carried out on 9, 10, 11, 12 and 13 day-old plants (the seedling were spread out over five days).

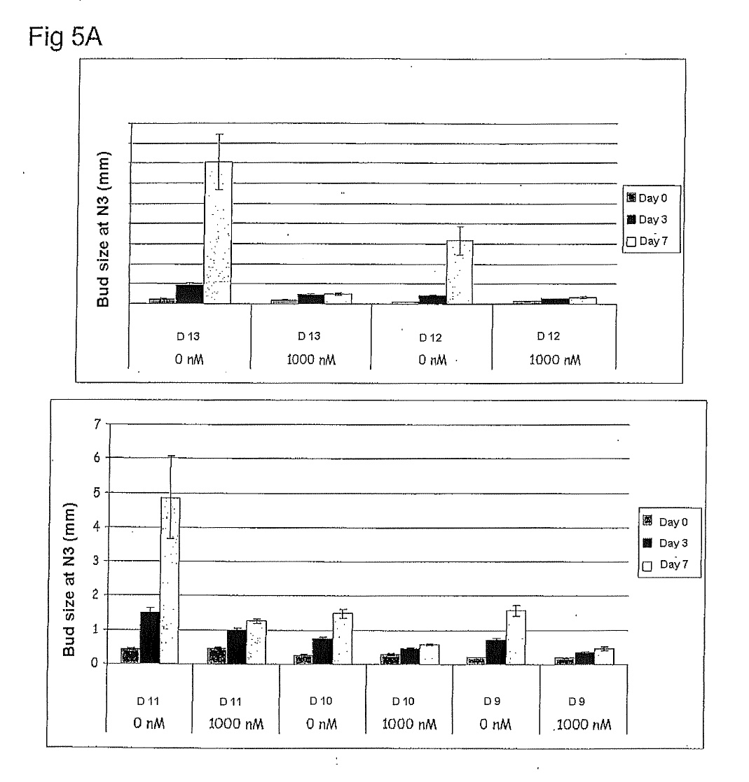

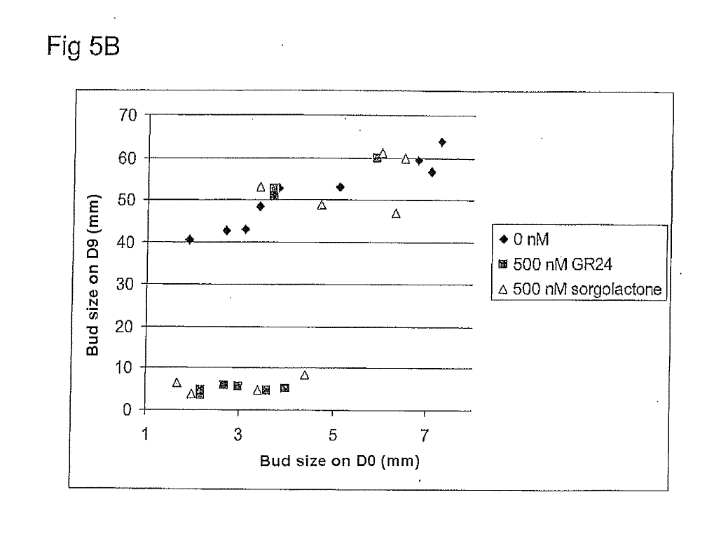

The bud size at the node N3 is measured the day of the first treatment (J0) and three and seven days afterwards. The obtained results are illustrated in the charts in FIG. 5A.

It is noted that all the buds, which are even so different ages, have a size between 0.2 and 1 mm the first day of treatment and are all sensitive to the treatment by direct application of GR24.

B] Experiment 2

A second experiment is carried out on mutants rms1 (line M3T-884 from WT Térèse) in order to compare the effect of synthetic strigolactones GR24 on the bud size at the time of the treatment.

The plants used are obtained in the same way as for the plants used in the preceding experiment.

The plants are treated by application of a solution (4% PEG 1450, 10% ethanol) containing sorgolactone or synthetic strigolactone GR24 at 0 nM and 500 nM. The solution is applied with the help of a micropipette onto the buds at the node 3 (N3), at the rate of 10 μl per bud.

The buds and/or ramifications at the first two plant nodes N1 and N2 are cut at the time of the treatment.

The size of the treated buds is measured nine days after the treatment. The chart in FIG. 5B shows the influence of the bud size at the time of the treatment (J0) on the effect of strigolactone on the treated bud.

It is noted that the bud size is limited to a threshold beyond which the buds are not anymore sensible to the treatment by application of strigolactones. Thus, in the experiment carried out here on peas and on this genotype, there is virtually no effect of stroglactones on the treated buds with a size of more than 4 to 5 mm at the time of the treatment.

### Example 3

### Test of Hyper-Ramification Pea Mutants with Injection of Strigolactones into the Stem for Proving the Long-Distance Action and the Effect Dose-Response of Strigolactones

The objective is here to show the effect of the injection of strigolactones at different concentrations into the plant stems, at nodes above the injection zone.

To this end, an experiment is carried out on mutants rms1 (line M3T-988 from WT Térèse) obtained in the same manner as for the mutants used in the preceding experiments.

The plants are treated by injecting the solution into the stem above the node N3. More precisely, a cotton thread is introduced into the plant stem with the help of needle and is dipped into the solution to be tested. The GR24 solution used (0 nM, 1 nM, 10 nM, 100 nM and 500 nM) were prepared by diluting in water GR24 solutions kept in acetone at different concentrations so that they have the same volume of acetone (10 μl of acetone in 20 ml of water).

The buds and/or ramifications at the first two plant nodes N1 and N2 are cut at the time of the treatment.

The “untreated” plants correspond to control plants the ramifications N1 and N2 of which have been cut, but which have not received any injection.

The size of the buds at the node situated a distance above the injection zone (N5) is measured eight days after the treatment.

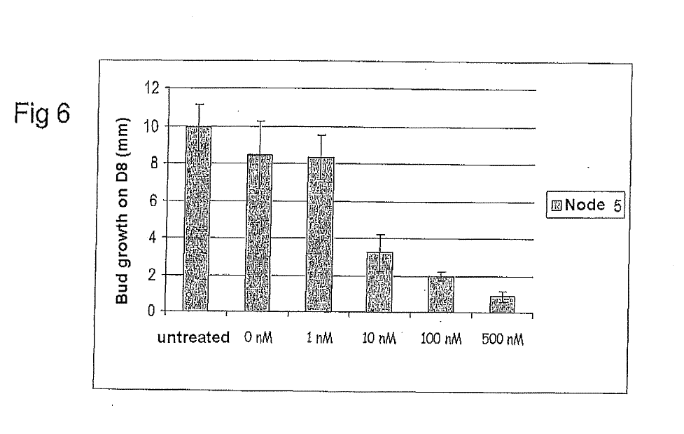

The chart in FIG. 6 shows the bud size at the node N5 eight days after the treatment, according to the treatment.

It is noted that the GR24 injection above N3 enables to repress the growth of the bud situated a certain distance from the injection zone (N5) right from 10 nM.

Strigolactone can thus have an action from a distance on the growth of axillary buds: it s probably carried in the xylem sap.

### Example 4

### Test of Hyper-Ramification Mutants of Peas Treated Before or after Decapitation

A] Experiment 1: Decapitation after Treatment

A first experiment is carried out simultaneously on wild peas (line WT Parvus) and mutants rms1 (line WL5237 from WT Térèse). In this experiment, 18 seeds are used for each treatment, which are sowed in pots (2 plants per pot with a diameter of 15 cm) in a soil mixed with sand. The seedling is carried out under glass with natural light and with an extension of the light period of 18 h light/6 h night be means of incandescent bulbs (60 W).

In a first stage, the plants are treated by two successive applications, separated by a 24 hour interval, of a solution (2% PEG 3550, 50% ethanol) containing synthetic strigolactone GR24 at 0 nM or 1000 nM. The solution is applied with the help of a micropipette onto the buds at the node 3 (N3), at the rate of 10 μl per bud.

The buds and/or ramifications at the first two plant nodes N1 and N2 are cut at the time of the treatment.

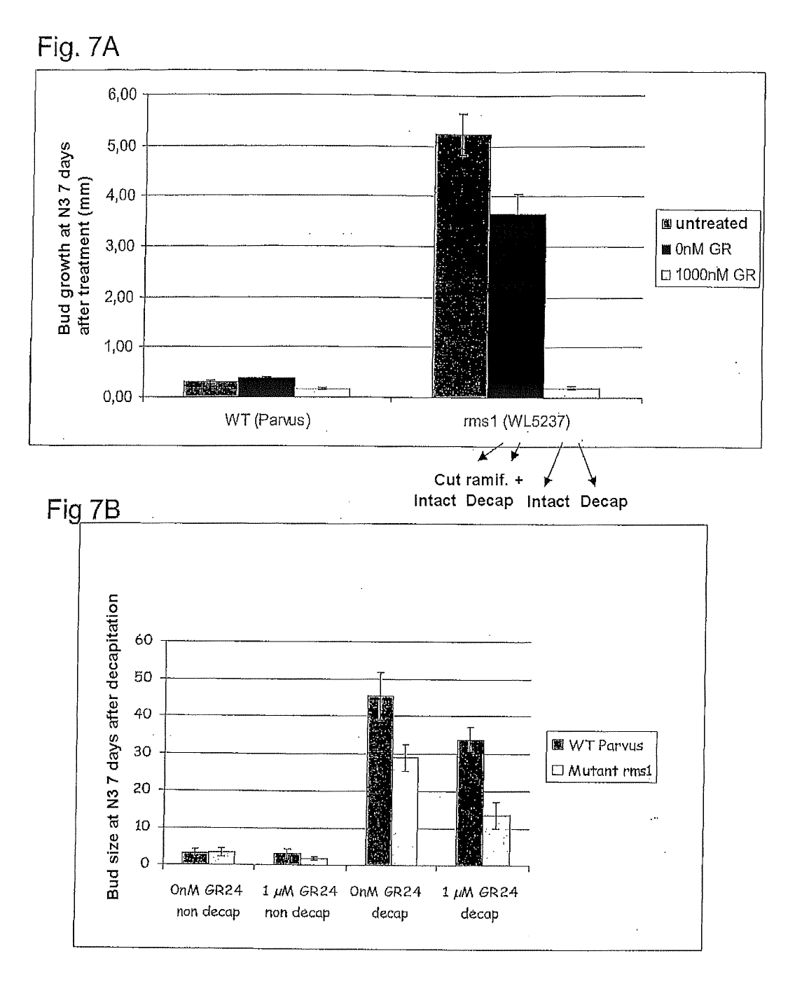

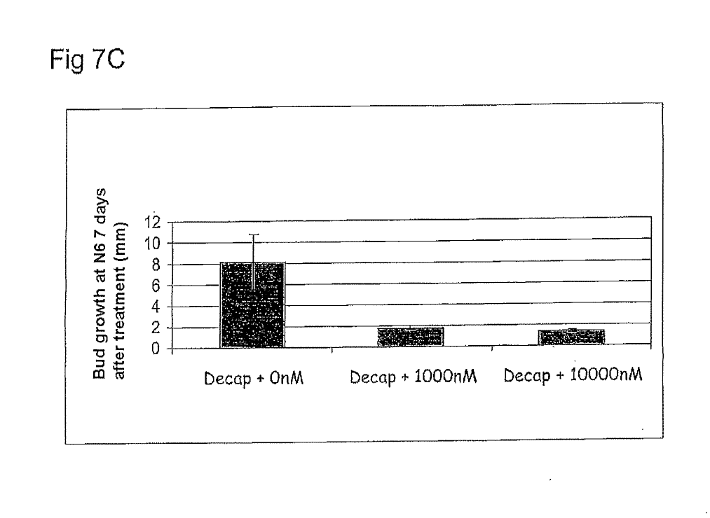

The size of the treated buds is measured seven days after the treatment. The chart in FIG. 7A illustrates the results obtained with the buds at the N3.

In a second stage, a first half of the treated mutant plants rms1 are decapitated just above the node 3, nine days after the treatment, the second half is left intact. The buds and/or ramifications at node 3 of the treated plants at 0 nM (which have not been repressed) are cut.

The bud size is measured seven days after the decapitation. The chart in FIG. 7B illustrates the results obtained with the buds at N3.

It is noted that the buds inhibited by GR24 in mutant rms1 are able to start again when the plant is decapitated, unlike the treated buds of the non-decapitated plants.

B] Experiment 2: Treatment with Decapitation

A second experiment is carried out with wild pea plants WT Torsdag obtained in the same manner as in the preceding experiment (the plants are in the stage with 6 nodes).

The buds at the plant node N6 are treated with four successive applications, separated by a 24 hour interval, of a solution (2% PEG 3550, 50% ethanol) containing strigolactone GR24 at 0 nM, 1000 nM or 10000 nM.

The buds and/or ramifications are cut at nodes N1 to N5 at the time of the treatment, whereas each plant is decapitated just above the node 6 just before the first application of the GR24 solution.

The buds at N6 are measured seven days after the first application, the results being shown in FIG. 7C.

It is noted that strigolactone enables, at least at high concentrations, to repress the start of axillary buds which had been even so induced and favored by a decapitation.

### Example 5

### Test of Hyper-Ramification Pea Mutants with Local Application of Strigolactones onto the Apical Bud

An experiment is carried out on wild pea plants WT Parvus, in order to observe the effect of strigolactone on the main stem.

The tested plants are obtained in the same manner as the plants of both preceding experiments.

The treatment is carried out 25 days after the seedling (with a development of about seven nodes).

2 μl of a solution of 0.1% silwet, at a GR24 concentration of 0 nM or 10000 nM, are applied onto the apical bud of each plant. As a control, plants are simultaneously treated by application of 1 μg of GA3 (1.44 mM) in 0.1% silwet on the apical bud to check that this treatment using silwet enables the penetration of hormones into the plant tissues.

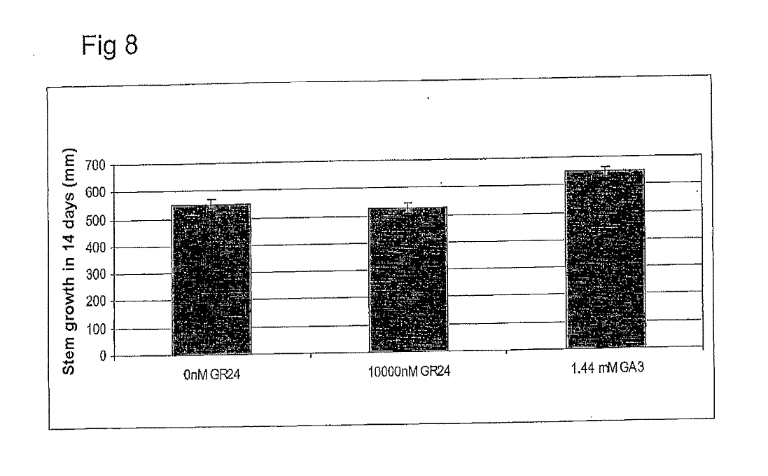

The size of the main stem is measured 14 days after the treatment, the results being represented in FIG. 8.

No effect of strigolactones on the growth of the main stem is noted, even at a high concentration. These results are in accordance with the results of the experiment carried out on buds of different ages in which the treatment is inefficient when buds have already started again.

Thus, strigolactone does not repress the growth of the apical bud and of the main stem, as well as of ramifications that have already started again and behave then as a main stem.

This observation enables to use strigolactones for controlling the growth of trees, such as oak, birch, beech etc which are cultivated for their wood, in order to limit the number of ramifications and to obtain trunks virtually without any big nodes.

### Example 6

### Test of Hyper-Ramification Mutants of Arabidopsis thaliana and of Wild Pants with Local Application of Strigolactones

An experiment, similar to that carried out with wild pea and mutant in the Example 1, is carried out with Arabidopsis thaliana, in order to prove that strigolactones are able to have an action on different plant species.

The plants used are obtained from wild lines WT Columbia, mutants max1 (mutant with the signal SMS in the stage of the biosynthesis pathway before both “Carotenoid Cleavage Dioxygenase”) and max2 (corresponding to response pea mutant rms4).

The plants are sowed in small containers and have been stocked at 4° C. for two days before being transferred in a conditioned chamber at 22° C. The plants are watered (sub-irrigation) every two days with an addition of nutriments every ten days. The daylight period is of 18 hours. A first GR24 treatment is carried out on the 23^{rd} day, just before the flowering. The number of treated plants varies between 25 and 41.

In total, the plant buds are treated with seven applications every three days over a period of 20 days: each treatment carried out with help of a micropipette consists in the application of 50 μl of a GR24 solution at 0 nM or 5000 nM in 0.1% Tween20. The application on the buds is carried out at the axil of rosette leaves or at the axil of buds that have already started.

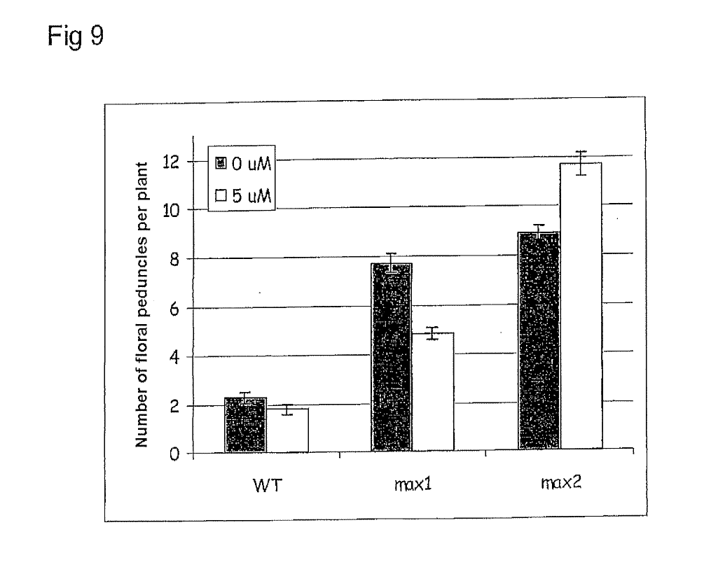

The number of floral peduncles is counted on 48 day-old plants, just before senescence. The results are shown in FIG. 9.

As in the case of pea, it is noted that strigolactone enables to repress ramifications in wild Arabidopsis as well as in mutant max1, but not in mutant max2.

The effect of strigolactone on ramifications is thus kept among the different species.

### Example 7

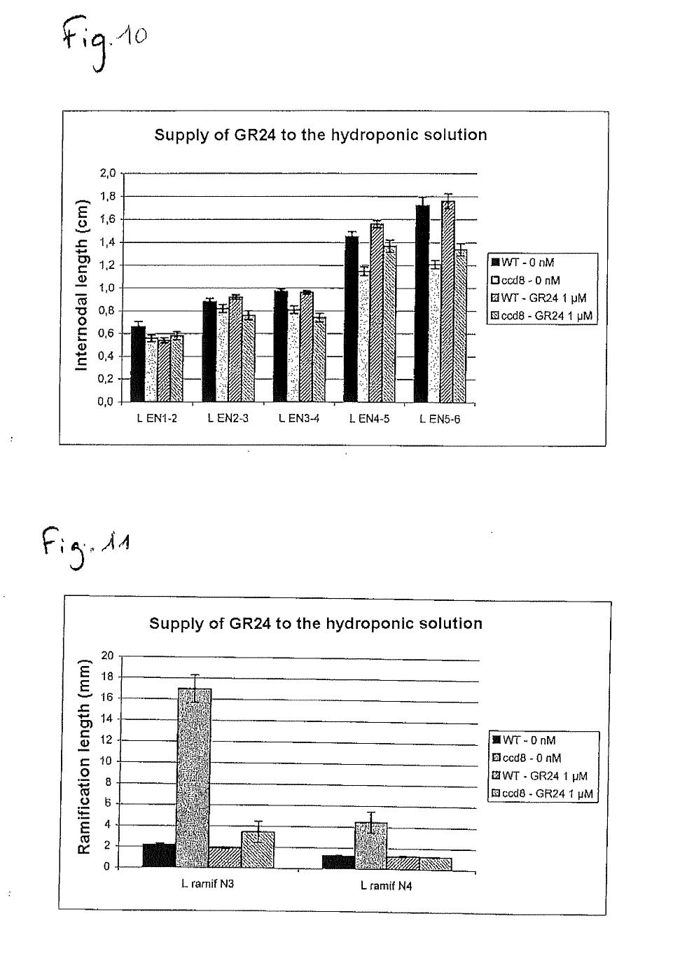

### Test of Hyper-Ramification Pea Mutants and of Wild Peas with Application of Strigolactones Through the Root to Prove the Positive Effect on the Plant Height (Internodal Length; FIG. 10) and the Inhibitive Effect on the Bud Start (FIG. 11)

The objective is to show the effect of the application of strigolactones, through the roots, on the buds and on the plant height.

To this end, an experiment is carried out in a hydroponic solution in order to bring GR24 into the hydroponic solution through the roots.

In this experiment, wild pea seeds (line WT Térèse) and mutant pea seeds (line M3T-988 ccd8/rms1 from WT Térèse) are first sowed in sand. Eight days later, the resulting plants are brought into the hydroponic system in which the roots are dipped in the nutritive solution. Four days later, a GR24 solution (diastereo isomer no 1) is added to 1 μM in the 47 liters of nutritive solution (4.7 ml GR24 at 10 mM). In this experiment, the plants reach the stage with 3-4 nodes.

Then, the cotyledonal ramifications are removed. The ramifications at the nodes N1 and N2 are kept.

The observation is carried out seven days later after addition of GR24 in the solution.

FIG. 10 represents the internodal length measured 19 days later after the germination on treated and untreated wild plants on one hand and treated and untreated mutant plants on the other hand.

It is noted that the addition of GR24 to the hydroponic solution has an effect only from the internode N4-N5. Under the internodes N1-N2, N2-N3 and N3-N4, GR24 has no action for these internodes are already developed before the addition of GR24.

FIG. 11 represents the ramification length (ramifications 3 at node N4) measured 19 days later on treated and untreated wild plants on one hand and treated and untreated mutant plants on the other hand. The ramifications at nodes N1 and N2 have already started at the time of the addition of GR24.

It is noted that the addition of GR24 to the hydroponic solution induces a reduction of the ramification length.

It is noted that the addition of strigolactones through the roots (dipped in the hydroponic solution) enables to increase the internode size. Moreover, it is noted that this very addition of strigolactones through the roots enables to inhibit the bud start.

Thus, the application of strigolactones though the roots would also enable to have an effect on the plant height.

### BIBLIOGRAPHIE

Beveridge C A (2000) Long-distance signalling and a mutational analysis of branching in pea. Plant Growth Regulation 32:193-203
Beveridge C A (2006): Axillary bud outgrowth: sending a message. Current Opinion in Plant Biology 9:35-40
Beveridge C A, Ross J J, Murfet I C. (1996) Branching in pea. Action of genes Rms3 and Rms4. Plant Physiol 110:859-865
Beveridge C A, Symons G M, Murfet, I C, Ross J J, Rameau C (1997) The rms1 mutant of pea has elevated of indole-3-acetic acid levels and reduced root-sap zeatin riboside content but increased branching controlled by graft transmissible signal(s). Plant Physiol 115: 1251-1258.
Booker J, Auldridge M, Wills S, McCarty D, Klee H, Leyser O (2004): MAX3/CCD7 is a carotenoid cleavage dioxygenase required for the synthesis of a novel plant signaling molecule. Curr Biol, 14:1232-1238.
Booker J, Sieberer T, Wright W, Williamson L, Willett B, Stirnberg P, Turnbull C, Srinivasan M, Goddard P, Leyser O (2005): MAX1 encodes a cytochrome P450 family member that acts downstream of MAX3/4 to produce a carotenoid-derived branch-inhibiting hormone. Dev Cell, 8:443-449.
Dharmasiri N, Dharmasiri S, Estelle M (2005) The F-box protein TIR1 is an auxin receptor. Nature 435:441-445
Foo E, Bullier E, Goussot M, Foucher F, Rameau C, Beveridge C A (2005) The branching gene RAMOSUS1 mediates interactions among two novel signals and auxin in pea. Plant Cell 17:464-474.
Foo E, Turnbull C G N, Beveridge C A (2001) Long distance signalling and the control of branching in the rms1 mutant of pea. Plant Physiol 126:203-209
Guzmán P, Ecker J R. (1990) Exploiting the triple response of Arabidopsis to identify ethylene-related mutants. Plant Cell. 6:513-23
Hayashi K, Tan X, Zheng N, Hatate T, Kimura Y, Kepinski S, Nozaki H. (2008) Small-molecule agonists and antagonists of F-box protein-substrate interactions in auxin perception and signaling. Proc Natl Acad Sci U S A 105:5632-5637
Johnson X, Brcich T, Dun E, Goussot M, Haurogné K, Beveridge C A and Rameau C (2006) Branching genes are conserved across species: genes controlling a novel signal in pea are co-regulated by other long-distance signals. Plant Physiol. 142: 1014-1026
Leyser H M, Lincoln C A, Timpte C, Lammer D, Turner J, Estelle M. (1993) Arabidopsis auxin-resistance gene AXR1 encodes a protein related to ubiquitin-activating enzyme E1. Nature, 364:161-164
Matusova R, Rani K, Verstappen F W, Franssen M C, Beale M H, Bouwmeester H J. (2005) The strigolactone germination stimulants of the plant-parasitic Striga and Orobanche spp. are derived from the carotenoid pathway. Plant Physiol 139:920-934
Morris S E, Colin G. N. Turnbull C G N, Ian C. Murfet I C, Beveridge C A (2001) Mutational Analysis of Branching in Pea. Evidence that Rms1 and Rms5 regulate the same novel signal. Plant Physiology 126:1205-1213.
Sorefan K, Booker J, Haurogné K, Goussot M, Bainbridge K, Foo E, Chatfield S, Ward S, Beveridge C, Rameau C, Leyser O (2003) MAX4 and RMS1 are orthologous dioxygenase-like genes that regulate shoot branching in Arabidopsis and Pea. Genes Dev. 17:1469-1474.
Stirnberg P, van de Sande K, Leyser HMO (2002): MAX1 and MAX2 control shoot lateral branching in Arabidopsis. Development, 29:1131-1141.
Thines B, Katsir L, Melotto M, Niu Y, Mandaokar A, Liu G, Nomura K, He S Y, Howe G A, Browse J. (2007) JAZ repressor proteins are targets of the SCF(COI1) complex during jasmonate signalling. Nature. 448:661-5
Ulmasov T, Hagen G, Guilfoyle T J. (1997) ARF1, a transcription factor that binds to auxin response elements. Science 276:1865-1868
Xie D X, Feys B F, James S, Nieto-Rostro M, Turner J G (1998) COI1 An Arabidopsis gene required for jasmonate-regulated defense and fertility. Science, 280:1091-1094

1. Treatment process for a superior plant in order to control the growth and architecture of the plant, characterized in that an adapted quantity of strigolactones is brought in contact with the plant so as to inhibit the formation of at least one ramification.

2. Treatment process according to claim 1, characterized in that strigolactones are brought in the form of a solution comprising natural and/or synthetic strigolactones, said synthetic strigolactones comprising GR24 and the molecule ABC.

3. Treatment process according to claim 1, characterized in that a solution comprising strigolactones is applied onto an at least partial portion of the aerial part of the plant.

4. Treatment process according to claim 1, characterized in that strigolactones are applied on axillary buds of the plant, so as to control the growth of the so-treated buds.

5. Treatment process according to claim 1, characterized in that a solution comprising strigolactones is injected into an aerial part of the plant so as to control the growth of the plant part above the injection zone.

6. Treatment process according to claim 2, characterized in that the strigolactone concentration in the composition is at least of 1 nM.

7. Treatment process according to claim 1, characterized in that a solution comprising strigolactones is brought through at least one root of the plant so as to control the ramification and/or the height of the plant.

8. Method of using strigolactones for identifying genes and/or molecules intervening in the control of the growth of axillary buds and/or ramifications in superior plants.

9. The method of using strigolactones according to claim 8, for identifying strigolatone receptors.

10. The method of using strigolactones according to claim 8, for identifying components of the signaling parthway for said strigolactones by screening mutants resisting to said strigolactones.

11. The method of using strigolactones according to claim 8, for identifying chemical analogs said strigolactones.

12. The method of using strigolactones according to claim 8, for identifying agonists and/or antagonists of said strigolactones.
